(19) United States
(12) Patent Application Publication
(10) Pub. No.: US 2022/0375548 A1
(43) Pub. Date: Nov. 24, 2022

(54) FRAGRANCE COMPOSITION TONALITY DETERMINATION METHOD, FRAGRANCE COMPOSITION DETERMINATION METHOD AND CORRESPONDING SYSTEMS

(71) Applicant: FIRMENICH SA, Satigny (CH)

(72) Inventors: Ben SMITH, Plainsboro, NJ (US); Patrick PFISTER, Plainsboro, NJ (US); Lily WU, Plainsboro, NJ (US); Hyo Young JEONG, Plainsboro, NJ (US); Daniel RAPS, Plainsboro, NJ (US); Maria BORISOVSKA, Plainsboro, NJ (US)

(21) Appl. No.: 17/634,080

(22) PCT Filed: Oct. 2, 2020
(86) PCT No.: PCT/EP2020/077721

§ 371 Date: Feb. 9, 2022

Related U.S. Application Data
(60) Provisional application No. 62/911,096, filed on Oct. 4, 2019.

(30) Foreign Application Priority Data
Nov. 28, 2019 (EP) 19212031.9

(51) Int. Cl. G16C 20/30; C11B 9/00

(52) U.S. Cl. CPC G16C 20/30; C11B 9/00

(57) ABSTRACT

The composition tonality determination method includes:

inputting at least one volatile molecule digital identifier, the volatile molecule digital identifier representative of a fragrant volatile molecule, the input defining a formula,

calculating, by a computing system, for at least one volatile molecule digital identifier of the formula, a value representative of an impact of each molecule on an activity level of an odorant receptor, represented by an odorant receptor digital identifier, each volatile molecule digital identifier being associated with at least one odorant receptor digital identifier, the association being a many-to-many association, and

determining, by a computing system, for the formula and as a function of at least one odorant receptor activity level impact calculated and a value representative of an odorant receptor activation threshold, a value representative of at least one tonality forming a composition, each odorant receptor digital identifier being associated with one tonality digital identifier, the association being a one-to-one association.

### CROSS-REFERENCE TO RELATED APPLICATIONS

This application is a U.S. National Phase Application of International Patent Application No. PCT/EP2020/077721, filed Oct. 2, 2020, which claims priority to European Patent Application No. 19212031.0, filed Nov. 18, 2019, and to U.S. Provisional Patent Application No. 62/911,096, filed Oct. 4, 2019, each of which is hereby incorporated by reference herein in its entirety.

### REFERENCE TO AN ELECTRONIC SEQUENCE LISTING

This application contains an electronic sequence listing. The contents of the electronic sequence listing (36803-335_Replacement_ST25.txt; Size: 46,455 bytes; Date of Creation: Jun. 23, 2022) is herein incorporated by reference in its entirety.

### TECHNICAL FIELD OF THE INVENTION

The present invention relates to a fragrance composition tonality determination method, a fragrance composition determination method, a fragrance composition tonality determination system and a fragrance composition determination system. It applies, in particular, to the fields of fragrance design and olfactometry, perfumery, fine fragrance perfumery and flavor design. This invention also applies to odorless or non-perfumistic compositions aiming to enhance or reduce the preceivability particular tonalities of other compositions.

### BACKGROUND OF THE INVENTION

Following the initial identification of odorant receptors in 1991, much has been done to advance the understanding of olfaction and how humans perceive volatile chemicals. At the periphery of the human olfactory system, the main olfactory epithelium lining the nasal cavity harbors several million olfactory sensory neurons (OSNs), each expressing a single member from a family of approximately 400 intact odorant (olfactory) receptor (OR) genes. Each OR can be activated by several volatile compounds while, in turn, a single volatile compound can also activate several ORs. This results in a combinatorial OSN activation code for each volatile chemical, and mixture thereof. Each receptor is stochastically expressed in OSNs in a monogenic and monoallelic fashion, yielding one OSN type for each OR allele present in the genome. Depending on the frequency of gene choice for each OR, the subset of the total OSN population dedicated to each OSN type varies. The OSN-type subpopulations each provide a discrete channel of information about the molecular identity, or identities, and concentrations of volatile compounds with which they are in contact at any given time. The level of activity induced in each OSN of each OSN type varies according to the concentration of volatile compounds to which its OR is receptive, as well as according to the binding and activation parameters associated with each OR-compound pair. Further, some OR-ligand pairs induce or enhance OSN activity, while others reduce or prevent OSN activity. This can occur competitively, where compounds compete to bind an OR, or non-competitively, where more than one compound can bind an OR simultaneously. The resulting combinatorial logic of these approximately 400 input channels (i.e. the peripheral olfactory system) is transformed by subsequent, downstream, neurological networks and results in our sense of smell.

The fragrance and flavor (F&F) industry is constantly in search for new ingredients, novel perfumery and flavor applications, improved sensory experiences, and compounds that are more stable, biodegradable and non-toxic.

Because an odorant or aroma molecule may interact with several olfactory receptors (ORs), it is often difficult to infer the tonality of volatile compounds based on its chemical structure alone. Conversely, because each OR may interact with several odorant and aroma molecules possessing different chemical structures and evoke different sets of tonalities, it has often been difficult to infer the nature of the information encoded by an OR.

There remains a need to predict the at least one olfactory tonality of a given compound or composition based on OR activation and to facilitate new perfumery and flavor ingredient discovery relevant to the F&F industry.

### SUMMARY OF THE INVENTION

The present invention is intended to remedy all or part of these disadvantages.

To this effect, according to a first aspect, the present invention aims at a composition tonality determination method, comprising:
a step of inputting at least one volatile molecule digital identifier, upon a computer interface, said volatile molecule digital identifier being representative of a fragrant volatile molecule, said input defining a formula,
a step of calculating, by a computing system, for at least one volatile molecule digital identifier of the formula, a value representative of an impact of each said molecule on an activity level of an odorant receptor, represented by an odorant receptor digital identifier, each volatile molecule digital identifier being associated with at least one odorant receptor digital identifier, said association being a many-to-many association and
a step of determining, by a computing system, for the formula and as a function of at least one odorant receptor activity level impact calculated and a value representative of an odorant receptor activation threshold, a value representative of at least one tonality forming a composition, each odorant receptor digital identifier being associated with one tonality digital identifier, said association being a one-to-one association.

Such provisions allow for the automatic prediction of the tonality of a dynamically input composition. Such a prediction allows for a more efficient and more productive fragrance and flavor design cycle.

In particular embodiments, the method object of the present invention comprises, downstream of the step of calculating, a step of computing, by a computing system, for at least one odorant receptor, a total activity level as a function of at least one impact on an activity level calculated.

Such provisions allow for the prediction of a total impact upon the preceivability of a tonality of a given composition as opposed to a molecule by molecule approach. In particular embodiments, the method object of the present invention comprises:
a step of modification, upon a computer interface, of a value representative of a fragrance tonality of the composition,
a step of calculation, by a computing system, of a modification of total activity level of at least one odorant receptor,
a step of determination, by a computing system, of a set of at least one volatile molecule digital identifier presenting a total activity level impact value equal to the total activity level modification calculated,
a step of supplying said set to a computer interface.

Such provisions allow for the automatic modification of the formula by fitting new molecules into the formula or by removing input molecules from the formula so that the target composition of at least one tonality is reached.

In particular embodiments, each tonality is associated to a tonality digital identifier, said method comprising a step of automatic tonality selection as a function of the result of comparing the tonality digital identifiers of the composition to a set of at least one predetermined tonality digital identifier, the step of modification of a value representative of a fragrance of said selected tonality identifiers of the composition being configured to reduce the value of said selected tonality identifiers to a reduced value, preferably to zero.

Such provisions allow for the automatic removal of tonalities deemed unpleasant or unnecessary.

In particular embodiments, the value representative of an impact of a molecule, represented by a volatile molecule digital identifier, on an activity level of an odorant receptor is representative of:
the activation of said odorant receptor,
the deactivation of said odorant receptor,
the enhancement of said odorant receptor and/or
the inhibition of said odorant receptor.

Such embodiments allow for the accurate definition of the types of impacts a volatile molecule may have upon an odorant receptor.

In particular embodiments, the step of determination is configured to provide at least one volatile molecule digital identifier presenting a value representative of an impact on the activity of an odorant receptor associated to a tonality modified during the step of modification, said impact corresponding either to the enhancement of said odorant receptor or to the inhibition of said odorant receptor.

Such tonality modifiers are relevant to the context of the perfumery application. For example, adding to a formula a volatile molecule which reduces the activity of a malodor receptor would reduce the perception of surrounding malodors (with applications to toilets, cat litter, deodorants, laundry etc.). Similarly, enhancing the activity of a receptor using an enhancer with faster release kinetics (e.g. a top note) may allow selective boosting of a base note tonality earlier in the fragrance experience than would typically occur. In particular embodiments, the method object of the present invention comprises:
a step of selecting a volatile molecule digital identifier of the formula, upon a computer interface,
a step of determination, by a computing system, of at least a set of at least one volatile molecule digital identifier presenting a value representative of an impact on an activity level of a odorant receptor equal to the value representative of the impact on the activity level on said odorant receptor of the selected volatile molecule digital identifier and
a step of supplying said set to a computer interface.

Such provisions allow for the automatic determination of replacement molecules that may be used to reformulate the formula. Such replacement molecules may have better environmental or cost impacts than the initially input molecules.

In particular embodiments, the method object of the present invention comprises:
a step of selecting at least two volatile molecule digital identifiers of the formula, at least two said volatile molecules being associated with the activation of at least two distinct odorant receptors, called “target receptors”,
a step of determination of one volatile molecule digital identifier presenting a value representative of an impact on an activity level of each target receptor equal to the value representative of the impact on the activity level on said odorant receptor of the selected volatile molecule digital identifiers and
a step of supplying the determined volatile molecule digital identifier to a computer interface.

Such provisions allow for the replacement of several molecules with a singular molecule, allowing for the optimization of the formula being designed.

In particular embodiments, the method object of the present invention comprises, downstream of either a step of determination, a step of estimation of a quantity for at least one said volatile molecule digital identifier, said quantity being used in a downstream step of supplying a set of at least one volatile molecule digital identifier.

Such provisions allow for a prediction of both the molecule to be added/removed from the formula and the quantity to add/remove of such molecule.

In particular embodiments, a step of determination, by a computing system, of at least a set of at least one volatile molecule digital identifier executes an optimization rule which is at least one of the following:
a first rule in which a determined set of at least one volatile molecule digital identifier is configured to present a minimal number of volatile molecule digital identifiers,
a second rule in which a determined set of at least one volatile molecule digital identifier is configured to present a minimal total quantity indicator of corresponding volatile molecule digital identifiers,
a third rule in which a determined set of at least one volatile molecule digital identifier is configured to present a minimal number of negative impact volatile molecule digital identifiers,
a fourth rule in which at least one volatile molecule digital identifier is associated to a volatile molecule delivery capacity indicator, said rule being configured to adapt a determined set of at least one volatile molecule digital identifier as a function of said volatile molecule delivery capacity indicator for the volatile molecule digital identifiers in the set,
a fifth rule in which a set of at least two volatile molecule digital identifier is configured to minimize the overlap of odorant receptors activated by said set of molecules,
a sixth rule in which a set of at least two volatile molecule digital identifier is configured to minimize the modulation of the odorant receptors activated by said set of molecules,
a seventh rule in which determined set of at least one volatile molecule digital identifier is configured to maximize the negative modulation or inhibition of odorant receptor linked to a predetermined undesired olfactory tonality,
an eighth rule in which determined set of at least one volatile molecule digital identifier is configured to maximize the positive modulation or enhancement of odorant receptor linked to a predetermined desired olfactory tonality and/or
a ninth rule in which determined set of at least one volatile molecule digital identifier is configured to minimize or maximize at least one cost function, each volatile molecule being associated to at least one value representative of cost. Such embodiments allow for the optimization of the formula following predetermined rules of optimization.

In particular embodiments, the method object of the present invention comprises a step of inputting quantity of at least one input volatile molecule, upon a computer interface, said quantity being used in the step of calculating, by a computing system, for at least one volatile molecule digital identifier of the formula, a value representative of an impact on an activity level of an odorant receptor.

Such embodiments allow for the consideration of quantity as a parameter of tonality prediction.

In particular embodiments, at least one volatile molecule digital identifier is associated with a volatile molecule delivery capacity indicator, at least one of steps of calculating or determination being performed as a function of the volatile molecule delivery capacity indicator associated to at least one input volatile molecule digital identifier.

Such embodiments allow for the more accurate determination of the tonality in a composition resulting from an input formula or, reversely, the accurate determination of the volatile molecules in a formula as a function of an input tonality.

In particular embodiments, the volatile molecule delivery capacity indicator is representative of a substrate upon which the associated volatile molecule is located.

In a second aspect, the present invention aims at a formula determination method, comprising:
a step of inputting, for at least one tonality digital identifier, upon a computer interface, a value representative of the tonality of a composition resulting from a formula to be determined,
a step of determining, by a computing system, for the formula to be determined and as a function of at least one value representative of at least one tonality of said composition, a value of odorant receptor activity level for at least one odorant receptor digital identifier representative of said odorant receptor, each odorant receptor digital identifier being associated with one tonality digital identifier, said association being a one-to-one association, and
a step of determination of a formula represented by at least a set of at least one volatile molecule digital identifier presenting a value representative of an impact on the activity level of said odorant receptor digital identifier equal to the determined value representative of the activity level on said odorant receptor, each volatile molecule digital identifier being associated with at least one odorant receptor digital identifier, said association being a many-to-many association.

The relationship between volatile molecule digital identifier and odorant receptor digital identifier may be one-to-many or many-to-one as well, in particular embodiments.

Such provisions allow for the reverse formula design wherein a user determines the tonalities to be reached and the formula is then deducted as a result of said target tonalities.

In a third aspect, the present invention aims at a composition tonality determination system, comprising:
a means of inputting at least one volatile molecule digital identifier, said volatile so molecule digital identifier being representative of a fragrant volatile molecule, said input defining a formula,
a means of calculating, for at least one volatile molecule digital identifier of the formula, a value representative of an impact on an activity level of each said molecule on an activity level of an odorant receptor, represented by an odorant receptor digital identifier, each volatile molecule digital identifier being associated with at least one odorant receptor digital identifier, said association being a many-to-many association and
a means of determining, for the composition and as a function of at least one odorant receptor activity level impact calculated and a value representative of an odorant receptor activation threshold, a value representative of at least one tonality forming a composition, each odorant receptor digital identifier being associated with at least one tonality digital identifier, said association being a one-to-one association.

Such a system provides the same advantages as the corresponding method. In a fourth aspect, the present invention aims at a formula determination system, comprising:
a means of inputting, for at least one tonality digital identifier, upon a computer interface, a value representative of the tonality of a composition resulting from a formula to be determined,
a means of determining, by a computing system, for the formula to be determined and as a function of at least one value representative of at least one tonality of said composition, a value of odorant receptor activity level for at least one odorant receptor digital identifier representative of said odorant receptor, each odorant receptor digital identifier being associated with one tonality digital identifier, said association being a one-to-one association, and
a means of determination of a formula represented by at least a set of at least one volatile molecule digital identifier presenting a value representative of an impact on the activity level of said odorant receptor digital identifier equal to the determined value representative of the activity level on said odorant receptor, each volatile molecule digital identifier being associated with at least one odorant receptor digital identifier, said association being a many-to-many association.

Such a system provides the same advantages as the corresponding method.

### BRIEF DESCRIPTION OF THE DRAWINGS

Other advantages, purposes and particular characteristics of the invention shall be apparent from the following non-exhaustive description of at least one particular method or system which is the object of this invention, in relation to the drawings annexed hereto, in which:

FIG. 1 represents, schematically, a first particular succession of steps of the method subject of the present invention,

FIG. 2 represents, schematically, a second particular succession of steps of the method subject of the present invention,

FIG. 3 represents, schematically, a first particular embodiment of the system subject of the present invention,

FIG. 4 represents, schematically, a second particular embodiment of the system subject of the present invention,

FIG. 5 represents an example of content of four perfumery accords (A, B, C and D) of ingredients that activate ORD1, OR8B3 and OR5AU1, and their respective composition ratio,

FIG. 6 represents the percent activity levels of the ingredients activating OR8D1, OR8B3 and OR5AU1.

FIG. 7 represents the odorant receptor activity induced by accords A and B validation by comparing a determined model and an in vitro control experiment.

FIG. 8 represents the congruency between perfumery evaluation for the celery tonality and to the sensory prediction from the model,

FIG. 9 represents the odorant receptor activity induced by accords A, C and D validation by comparing the model and the in vitro control experiment,

FIG. 10 represents the congruency between perfumery evaluation for the hay tonality and to the sensory prediction from the model and

FIG. 11 represents the congruency between perfumery evaluation for the coconut tonality and to the sensory prediction from the model.

### DETAILED DESCRIPTION OF THE INVENTION

This description is not exhaustive, as each feature of one embodiment may be combined with any other feature of any other embodiment in an advantageous manner.

It should be noted at this point that the figures are not to scale.

The content of U.S. 62/911,096 is incorporated herein by reference.

As used herein, the terms “volatile molecule” designate any molecule, preferably presenting a flavoring or fragrance capacity. The terms “compound” or “ingredient” designate the same items as “volatile molecule.”

The term “formula” designates a liquid, solid or gaseous assembly of at least one volatile molecule.

The term “composition” refers to the olfactory perception of a formula. A composition is defined by the perceivability of at least one tonality.

As used herein, “olfactory tonality” or “tonality” means an organoleptic property of a volatile molecule initiated by the activation of an OR, which activity is routed through the olfactory bulb and processed by the central nervous system to produce a specific experience in the subject. Said experience is of a nature that may be described by the subject linguistically, often with reference to everyday objects with which the olfactory experience is associated. Additional modifiers are typically required to capture the specificity of the tonality. Everyday objects that possess a characteristic “smell” typically elicit the experience of multiple separable tonalities when evaluated by a trained expert such as a perfumer. The experience of a single tonality is entirely dependent on the specific OR activated in the subject, such that a single OR produces the perception of a single tonality and the multitude of tonalities produced by may everyday objects is determined by the set of ORs activated by the set of volatile molecules they emit. Though the mechanism differs in many important ways, the perception of an olfactory tonality may be considered analogous to the perception of a single discernable musical note through the auditory system, discernable even when multiple notes are played at once. Similarly, where the same musical note may be described in multiple ways (F-sharp, G-flat, etc.), so the same olfactory tonality may be described in multiple ways.

Non-limiting examples of an olfactory tonality include earthy, coconut, celery, hay etc. (see Example 1 in U.S. 62/911,096). For example, OR11A1 agonists all share a common earthy tonality even as they elicit an overall set of distinct tonalities (see Example 2 in U.S. 62/911,096). A volatile molecule may have greater than one tonality. For example, beta ionone has violet and blonde woody tonalities arising from the activation of OR5A1 and OR7A17, respectively (see Example 3 in U.S. 62/911,096).

As used herein, a “fragrance” refers to the olfactory perception resulting from the sum of odorant receptor(s) activation, enhancement and inhibition (when present) by at least one volatile molecule. Accordingly, by way of illustration and by no means intending to limit the scope of the present disclosure, a “fragrance” results from the olfactory perception arising from the sum of a first volatile molecule that activates an OR associated with a coconut tonality, a second volatile molecule that activates an OR associated with a celery tonality, and a third volatile molecule that inhibits an OR associated with a hay tonality.

The inventors have found that the activation of one OR triggers the perceivability of one tonality in one to one relationship so that a composition, being an assembly of at least one tonality, can be encoded by a set of corresponding activated ORs, potentially with an associated activation level.

As used herein, a “note” or “olfactory note” or “perfumery note” identifies a tonality category. For example, floral notes include muguet and violet tonalities.

The terms “OR” or “olfactory receptor” or “odorant receptor” refers to one or more members of a family of G protein-coupled receptors (GPCRs) that are expressed in olfactory cells. OSNs (olfactory sensory neurons) can also be identified on the basis of morphology or by the expression of proteins specifically expressed in olfactory cells. OR family members may have the ability to act as receptors for olfactory signal transduction.

An “agonist of an OR” or “agonist compound” refers to a volatile compound or a ligand that binds to an OR, activates the OR and induces an olfactory receptor transduction cascade.

An “antagonist of an OR” or “antagonist compound” refers to a volatile compound or a ligand that binds to an ORand reduces the measured activity of an olfactory receptor in the presence of an agonist.

An “enhancer” refers to a compound or ligand that binds to an OR and that enhances the activity of the receptor through an olfactory receptor transduction cascade exposed to the agonist compound when compared to the activity of the receptor exposed to the agonist compound in the absence of the enhancer. In one aspect, the compound that is not an agonist binds to a site on the receptor different from the site where the agonist compound binds.

“Potency” refers to the measure of the activity of a receptor induced by the binding of an agonist (odorant) in terms of amount or concentration required to produce a given activity level. It indicates the sensitivity of the receptor to different agonist concentrations and is often assessed by calculating the EC50 (the agonist concentration necessary to achieve half-maximal receptor activity).

“Efficacy” refers to the measure of the activity level by which the OR responds to a given agonist and is obtained by measuring the activation span between constitutive (baseline in the absence of an agonist) and agonist induced activity.

The “receptive field” or “molecular receptive range” of an odorant receptor refers to the range of volatile compounds activating the receptors. It describes the set of distinct volatile compounds able to bind to the receptor, trigger a transduction cascade within a cell, and hence transfer the chemical stimulus through the olfactory sensory neuron to the brain.

“OR” or “odorant receptor” or “olfactory receptor” polypeptides are considered as such if they pertain to the 7-transmembrane-domain G protein-coupled receptor superfamily encoded by a single approximately 1 kb long exon and exhibit characteristic olfactory receptor-specific amino acid motifs. The seven domains are called “transmembrane” or “TM” domains TM I to TM VII connected by three “internal cellular loop” or “IC” domains IC I to IC III, and three “external cellular loop” or “EC” domains EC I to EC III. The motifs and the variants thereof are defined as, but not restricted to, the MAYDRYVAIC motif (SEQ ID NO: 7) overlapping TM III and IC II, the FSTCSSH motif (SEQ ID NO: 8) overlapping IC III and TM VI, the PMLNPFIY motif (SEQ ID NO: 9) in TM VII as well as three conserved C residues in EC II, and the presence of highly conserved GN residues in TM I [Zhang, X. & Firestein, S. Nat. Neurosci. 5, 124-133 (2002); Malnic, B., et al. Proc. Natl. Acad. Sci. U.S.A 101, 2584-2589 (2004)].

“Delivery capacity” refers to the factor which converts the quantity of a volatile molecule in a formula to the quantity delivered to the olfactory receptors in the nose of a subject.

It should be understood that in the context of the present invention, two key relationships are established:
a first relationship, linking volatile molecule to the activation of ORs and
a second relationship, linking the activation of ORs to specific tonalities.

In particular, a given volatile molecule may interact with several ORs and one OR may interact with several volatile molecules. However, the activity of one OR may correspond to one perceived tonality triggered by the activity of one given OR. The perception of a single compound exhibiting several tonalities is resulting from the activation of several ORs encoding said tonalities. This “one-to-one” relationship is not trivial to discover and has major implications in formula and fragrance design. Indeed, in systems in which the volatile molecule to tonality is known only, it is impossible to accurately predict or estimate the tonality of a combination of volatile molecules, given the lack of knowledge of the intermediate layer of interaction between molecule and OR on one side and OR to tonality on another.

The first relationship may be obtained by putting into contact a sample of volatile molecules with isolated ORs and by monitoring the activity of each said OR. The result of such an experiment may be stored inside a database cross referencing volatile molecule digital identifiers, representative of real volatile molecules, and the impact of said volatile molecule on the ORs monitored during the experiment.

In such methods, as long as the function of an OR is not impaired, it may be used in any form in a method or assay described herein. For example, the OR may be used as follows: tissues or cells which intrinsically express an OR such as olfactory sensory neurons isolated from organism and cultured products thereof; olfactory cell membrane bearing the OR; recombinant cells genetically modified so as to express the OR and cultured products thereof; membrane of the recombinant cells; and artificial lipid bilayer membrane carrying the OR.

Indicators for monitoring the activity of olfactory receptors include, for example, a fluorescent calcium indicator dye, a calcium indicator protein (e.g. GCaMP, a genetically encoded calcium indicator), a fluorescent cAMP indicator, a cell mobilization assay, a cellular dynamic mass redistribution assay, a label-free cell based assay, a cAMP response element (CRE) mediated reporter protein, a biochemical cAMP HTRF assay, a beta-arrestin assay, or an electrophysiological recording. In a particular embodiment, a calcium indicator dye is selected that can be used to monitor the activity of olfactory receptors expressed on the membrane of the olfactory neurons (e.g., Fura-2 AM). Molecules may be screened sequentially and the odorant-dependent changes in calcium dye fluorescence are measured using a fluorescent microscope or fluorescent-activated cell sorter (FACS).

Such methods allow the identification of commonalities in olfactory perception between chemically and organoleptically diverse volatile compounds based on their OR activation profile. Without intending to be limited to any particular theory, OR activation profiles represent better predictors of olfactory tonality for a given volatile compound, compared to physicochemical similarities alone. Without intending to be limited to any particular theory, physiochemical similarity may only partially predict olfactory similarity.

As an example, olfactory neurons activated by the target agonist, e.g. a molecule with a particular olfactive tonality, are isolated using either a glass microelectrode attached to a micromanipulator or a FACS machine. Mouse olfactory sensory neurons are screened by Ca2+ imaging similar to procedures previously described. Malnic, B., et al. Cell 96, 713-723 (1999); Araneda, R. C. et al. J. Physiol. 555, 743-756 (2004); and WO2014/210585. Particularly, a motorized movable microscope stage is used to increase the number of cells that can be screened to at least 1,500 per experiment. Since there are approximately 1,200 different olfactory receptors in the mouse and each olfactory sensory neuron expresses only 1 of 1,200 olfactory receptor genes, this screening capacity will cover virtually the entire mouse odorant receptor repertoire. In other words, the combination of calcium imaging for high-throughput olfactory sensory neuron screening leads to the identification of nearly all of the odorant receptors that respond to a particular profile of odorants. In one aspect, odorant receptors that respond to the target agonist, e.g. with a particular olfactive tonality, can be isolated for receptor identification. For example, at least one neuron is isolated for receptor identification.

The second relationship may be obtained by associating at least one olfactive tonality with an OR by screening chemically and organoleptically diverse libraries of volatile compounds against the OR to identify the at least one olfactive tonality common to the volatile compounds that are activators of the screened ORs.

Such a method for associating at least one olfactory tonality to an olfactory receptor can be obtained by providing an OR, contacting the OR and a volatile molecule having a known at least one tonality, determining whether the compound activates the at least one olfactory receptor, repeating the contacting and determining steps with different compounds having known at least one tonality, categorizing a subset of compounds that activate the OR, identifying an at least one known tonality common to the subset of compounds, and associating the identified at least one known tonality to the OR.

The common olfactive tonality of compounds activating a given receptor can be associated by comparing the overall description of the compounds and identifying the descriptor that is common among activators. It should be understood that such an olfactive tonality can be semantically described, e.g., by a perfumer, in several ways. Examples of such semantic similarities capturing the same olfactive tonality include for example: marine, watery and ozone; earthy, humus and moss; hay, coumarinic and tonka; celery, fenugreek and maple; muguet and lily-of-the-valley.

The result of such an experiment may be stored inside a database cross referencing OR digital identifiers, representative of real ORs, and information representative of the tonalities associated to the activation of these ORs.

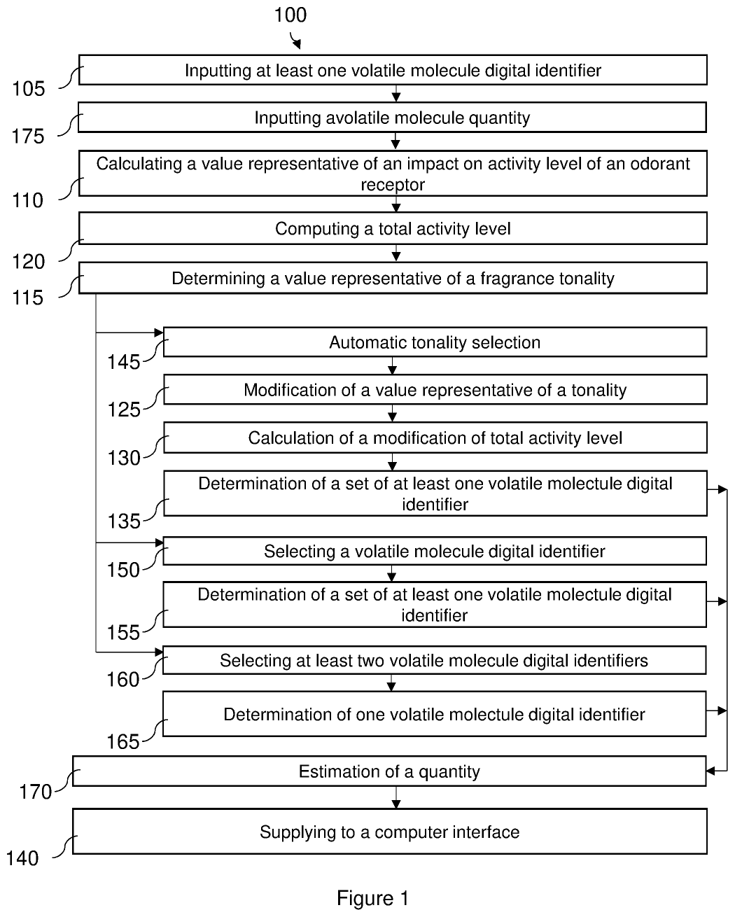

FIG. 1 shows a particular succession of steps of the method 100 object of the present invention. This composition tonality determination method 100, comprises:
a step of inputting 105 at least one volatile molecule digital identifier, upon a computer interface, said volatile molecule digital identifier being representative of a fragrant volatile molecule, said input defining a formula,
a step of calculating 110, by a computing system, for at least one volatile molecule digital identifier of the formula, a value representative of an impact of each said molecule on an activity level of an odorant receptor, represented by an odorant receptor digital identifier, each volatile molecule digital identifier being associated with at least one odorant receptor digital identifier, said association being a many-to-many association and
a step of determining 115, by a computing system, for the formula and as a function of at least one odorant receptor activity level impact calculated and a value representative of an odorant receptor activation threshold, a value representative of at least one tonality forming a composition, each odorant receptor digital identifier being associated with one tonality digital identifier, said association being a one-to-one association.

The step of inputting 105 may be performed using any type of computer interface, such as a keyboard, a mouse or a touchscreen for example. Such interface may further comprise a graphical user interface (GUI) allowing for user interaction and input, said GUI being part of a software ran by a computing means, such as a personal computer or computer server. In variants, the computer interface is logical in nature, the input corresponding to a command received through an electronic network or cable and originating from a command means.

The particular architecture of the computing system used in FIGS. 1 to 4 is unimportant with regards to the present invention. That is to say, such a computing system may be distributed, integrated, using a client-server architecture or using local and/or distant computing resources. Data stored and accessed may be stored in traditional databases, in computer memories or in distributed databases.

During this step of inputting 105, a user may select one or more volatile molecule digital identifier to add in a formula. A volatile molecule digital identifier may be an icon, a text label or a number for example. Such a volatile molecule digital identifier corresponds, preferably, to an entry into a computer memory or a database.

A formula may further comprise a quantity of volatile molecule, expressed either in liquid phase, solid phase or gaseous phase quantities. Such a quantity may be expressed in parts per million (ppm) or in a measure of headspace concentration of the gaseous phase of said molecule, in mol/m³, for example.

The resulting assembly of tonalities perceived from the evaporated and/or gaseous form of the volatile molecules, and the transportation of the volatile molecules is called a composition.

The step of calculating 110 is performed, for example, by a computing system configured to run a dedicated software. During this step of calculating 110, each input volatile molecule digital identifier is matched with the corresponding activated OR. If an OR is activated or enhanced, the impact on that OR is positive. If an OR is deactivated, or inhibited from activation, the impact on that OR is negative. One volatile molecule digital identifier may activate/enhance/deactivate/inhibit several ORs concurrently.

This step of calculating 110 may comprise a step (not represented) of accessing an information storage, such as a database or computer memory, in which the impact of a volatile molecule digital identifier on at least one referenced OR is stored and retrieving said information.

Such a step of calculating 110 is illustrated in Example n° 2 of U.S. patent application n° 62/911,096.

The step of determining 115 is performed, for example, by a computing system configured to run a dedicated software. During this step of determining 115, an information relative to the tonality associated to at least one OR activated by at least one volatile molecule is produced. Such an information may be the existence of said tonality or the efficacy of said OR on said tonality.

This means that the values representative of a tonality may represent:
the perceivability of one or more tonality,
the quantification of said perceivability,
the inhibition of said tonality,
the increase in perceivability of said tonality and/or
the reduction of said tonality.

This step of determining 115 may comprise a step (not represented) of accessing an information storage, such as a database or computer memory, in which the tonality information to be produced, for at least one OR, is stored and retrieving said information.

The step of determining 115 may comprise computing a value relative to the total activity of the at least one OR wherein the at least one value is representative of a quantification of the stored tonality. Such a value may indicate whether to retrieve the associated tonality, the perceived intensity of the tonality, or the probability that the tonality is perceived by a subject, for example.

Such an information may be obtained by reading said information in the proper information storage or be the result of a computation by the computing system, for example.

Preferably, an OR activation level threshold is used in order to determine the value representative of a tonality such as disclosed above. In such variants, the activation threshold may be common for all ORs or determined on a OR by OR basis, said threshold being observed and recorded empirically. Such a threshold value is, for example, 20% of the average maximum activation level of ORs. Such threshold values are, for example, stored in an activation threshold database used during the step of determining 115.

The OR activity is dependent on potency and efficacy of any given agonist for this OR, that is obtained from receptor screening experiments. With this information, one can determine the minimum amount (based on potency data) of material needed to generate enough activity (based on efficacy data) to generate the perception of the corresponding tonality. The amount of a compound inhibiting or enhancing an OR can be similarly obtained from receptor screening data. This quantification can be adjusted to the target outcome (presence of a desired tonality, reduction of an undesired tonality) for each OR presented with a compound mixture (or formula).

The output of the step of determining 115 may be the supply of the resulting information upon a computer interface, such as a computer screen for example.

In particular embodiments, such as the one represented in FIG. 1, the method 100 object of the present invention comprises, downstream of the step of calculating 110, a step of computing 120, by a computing system, for at least one odorant receptor, a total activity level as a function of at least one impact on an activity level calculated.

The step of computing 120 is performed, for example, by a computing system configured to run a dedicated software. During this step of computing 120, the sum of individual impacts of the activated/enhanced/inhibited/deactivated ORs is obtained that the result is then used during the step of determining 115 to provide information.

In particular embodiments, such as the one represented in FIG. 1, the method 100 object of the present invention comprises:
a step of modification 125, upon a computer interface, of a value representative of a fragrance tonality of the formula,
a step of calculation 130, by a computing system, of a modification of total activity level of at least one odorant receptor,
a step of determination 135, by a computing system, of a set of at least one volatile molecule digital identifier presenting a total activity level impact value equal to the total activity level modification calculated,
a step of supplying 140 said set to a computer interface.

The step of modification 125 is performed, for example, similarly to the step of inputting 105 by collecting a user input via a human machine interface, said input being representative of the modification of a value representative of a fragrance tonality of the formula initially input. Said modification may be part of a formula modification command received electronically by the computing system.

The tonality modification can be the reduction of the degree of perceivability of a given tonality or the increase of the degree of perceivability of a given tonality. The step of calculation 130 is performed, for example, by a computing system configured to run a dedicated software. During this step of calculation 130, the modification affecting at least one tonality is translated into a modification of activity level for the underlying OR or ORs.

The step of determination 135 is performed, for example, by a computing system configured to run a dedicated software. During this step of determination 135, the modification of activity level for the OR or ORs calculated during the step of calculation 130 is translated into a modification of the input of volatile molecules activating/enhancing/inhibiting/deactivating said OR or ORs.

It should be understood that the total activity level impact value equal to the total activity level modification calculated is understood as a value located within an interval representative of an acceptable deviation from an exact equality.

Such a modification may correspond to:
the removal of a volatile molecule from the formula, translated into the removal of the corresponding digital identifier from the formula,
the addition of a volatile molecule to the formula, translated into the addition of the corresponding digital identifier from the formula and/or
the modification of a quantity of volatile molecule in the formula, translated into the modification of an indicator representative of the quantity of a corresponding digital identifier in the formula.

Given the fact that volatile molecule to OR activity impact is a many-to-many relationship and that OR activity impact to tonality perceivability is a one-to-one relationship for each OR and corresponding tonality, there are several combinatory solutions to the steps of calculation 130 and determination 135.

In order to determine an optimal resolution of these steps, optimization resolution rules are preferably set in place. Such resolution rules may be:
a first rule in which a determined set of at least one volatile molecule digital identifier is configured to present a minimal number of volatile molecule digital identifiers,
a second rule in which a determined set of at least one volatile molecule digital identifier is configured to present a minimal total quantity indicator of corresponding volatile molecule digital identifiers,
a third rule in which a determined set of at least one volatile molecule digital identifier is configured to present a minimal number of negative impact volatile molecule digital identifiers,
a fourth rule in which at least one volatile molecule digital identifier is associated to a volatile molecule delivery capacity indicator, said rule being configured to adapt a determined set of at least one volatile molecule digital identifier as a so function of said volatile molecule delivery capacity indicator for the volatile molecule digital identifiers in the set,
a fifth rule in which a set of at least two volatile molecule digital identifier is configured to minimize the overlap of odorant receptors activated by said set of molecules,
a sixth rule in which a set of at least two volatile molecule digital identifier is configured to minimize the modulation of the odorant receptors activated by said set of molecules,
a seventh rule in which determined set of at least one volatile molecule digital identifier is configured to maximize the negative modulation or inhibition of odorant receptor linked to a predetermined undesired olfactory tonality,
an eighth rule in which determined set of at least one volatile molecule digital identifier is configured to maximize the positive modulation or enhancement of odorant receptor linked to a predetermined desired olfactory tonality and/or
a ninth rule in which determined set of at least one volatile molecule digital identifier is configured to minimize or maximize at least one cost function, each volatile molecule being associated to at least one value representative of cost.

The fourth rule may represent the availability of the volatile molecules in the headspace to produce the desired level of activation. The rules account for the non-linear factors associated with delivery of a composition that affect the quantities reaching the ORs. Examples of these factors are deposition on substrates, evaporation from substrates, sorption rates into the mucus, trafficking by dedicated molecules in the mucus and biodegradation of molecules in the mucus.

In particular embodiments, at least one volatile molecule digital identifier is associated to a volatile molecule delivery capacity indicator, the step of calculating 110 being performed as a function of the volatile molecule delivery capacity indicator associated to at least one input volatile molecule digital identifier.

The volatile molecule delivery capacity indicator is stored within a volatile molecule delivery capacity indicator database and determined empirically, for example.

In one example, to determine a delivery capacity indicator, a formula containing at least one volatile molecule is dosed onto a relevant substrate at a given concentration, such as fabric, bare skin, a kitchen tile, cat litter, or aerosolized directly into the air, placed under relevant environmental conditions, such as in a chamber at a certain temperature, pressure and humidity, and left for a relevant amount of time. A known volume of the air within the chamber can be sampled by suitable equipment for a known period of time, such as sorbent tube or fiber, which could subsequently be analyzed by a quantitative chemical analytical instrument such as a Gas Chromatography Mass Spectrometer and the vapor phase concentrations of the at least one molecule could be determined.

In another example, known vapor phase concentrations of at least one volatile molecule representing a formula could be established in a suitable chamber containing an interface, of known surface area, with a known volume of a liquid phase substrate, such as mucus, for a defined period of time under suitable pressure, temperature and humidity. The concentrations of the at least one molecule can subsequently be determined by applying a sample of the liquid-phase substrate to a quantitative analytical chemistry instrument such a Liquid Chromatography Mass Spectrometer.

In a third example calculation of more complex delivery capacity indicators is possible by combining the prior two examples and/or by measuring physical chemical constants, such as the Henry constant, by established methods and combining them with relevant physics models.

The step of calculating 110 can, for example, be configured to compute the loss due to the molecule delivery capacity indicator, said loss impacting the value representative of the tonality determined in the step of determining 115.

In embodiments in which the value representative of tonality is set, a step of determination, 135, 155 or 165, may be performed as a function of the molecule delivery capacity indicator. In such embodiments, the loss due to the molecule delivery capacity indicator for a given volatile molecule digital identifier can be compensated by, for example, increasing the quantity of said volatile molecule in the formula.

In particular embodiments, the volatile molecule delivery capacity indicator is representative of a substrate upon which the associated volatile molecule is located.

Such a substrate is, for example, a type of cloth, a type of food product or the absence of such a substrate, such as the aerosolization of a perfume into the air.

Additional rules, relative to extrinsic parameters may be put in place. In variants, each volatile molecule digital identifier is associated to a cost, being an environmental, financial or other cost.

In a first example of such additional rules, a corresponding rule may be for a determined set of at least one volatile molecule digital identifier to be configured to present the minimal cost value.

In a second example of such additional rules, a corresponding rule may be for a determined set of at least one volatile molecule digital identifier to be configured to present a cost value inferior or superior to a predetermined threshold value.

In a third example of such additional rules, a corresponding rule may be for a determined set of at least one volatile molecule digital identifier to be configured to present a minimal sum of cost values, wherein at least two types of costs are considered.

The step of supplying 140 the result of the step of determination 135 corresponds to the step of supplying 140 discussed above.

In particular embodiments, such as the one shown in FIG. 1, each tonality is associated to a tonality digital identifier, said method comprising a step of automatic tonality selection 145 as a function of the result of comparing the tonality digital identifiers of the formula to a set of at least one predetermined tonality digital identifier, the step of modification 125 of a value representative of a fragrance of said selected tonality identifiers of the formula being configured to reduce the value of said selected tonality identifiers to a reduced value, preferably to zero.

The step of automatic tonality selection 145 is performed, for example, by a computing system configured to run a dedicated software. In this step of automatic tonality selection 145, the objective is to remove the unpleasant tonalities from a composition. Such unpleasant tonalities may correspond to specific predetermined tonality identifiers stored in a computer memory or database. During this step of automatic tonality selection 145, the tonalities determined during the step of determining 115 can be matched against the predetermined unpleasant tonality identifiers and at least one said matched tonality be modified, during the step of modification 125, be set to a reduced value, preferably to zero.

In particular embodiments, such as the one shown in FIG. 1, the method 100 object of the present invention comprises:
a step of selecting 150 a volatile molecule digital identifier of the formula, upon a computer interface,
a step of determination 155, by a computing system, of at least a set of at least one volatile molecule digital identifier presenting a value representative of an impact on an activity level of a odorant receptor equal to the value representative of the impact on the activity level on said odorant receptor of the selected volatile molecule digital identifier and
a step of supplying 140 said set to a computer interface.

The step of selecting 150 is performed, for example, in a similar manner to the step of inputting 105. This step of selecting 150 can be performed manually or automatically, as the result of a selection command transmitted to the computing system. The criteria for the selection can be arbitrary or follow an algorithmic resolution based upon information associated to each volatile molecule digital identifier, such as a cost value for each volatile molecule digital identifier.

The step of determination 155 is performed, for example, by a computing system configured to run a dedicated software. This step of determination 155 can be performed in a similar manner to the step of determination 135 discussed above. Such a step of determination 155 can follow optimization rules as discussed above.

This step of determination 155 allows for the replacement of a volatile molecule in the formula by at least one other volatile molecule, represented by the corresponding digital identifier or digital identifiers.

The step of supplying 140 the result of the step of determination 155 corresponds to the step of supplying 140 discussed above.

In particular embodiments, such as the one shown in FIG. 1, the method 100 object of the present invention comprises:
a step of selecting 160 at least two volatile molecule digital identifiers of the formula, at least two said volatile molecules being associated with the activation of at least two distinct odorant receptors, called “target receptors”,
a step of determination 165 of one volatile molecule digital identifier presenting a value representative of an impact on an activity level of each target receptor equal to the value representative of the impact on the activity level on said odorant receptor of the selected volatile molecule digital identifiers and
a step of supplying 140 the determined volatile molecule digital identifier to a computer interface.

Such embodiments aim for the simplification of the formula in terms of number of volatile molecules used.

The step of selecting 160 is performed, for example, in a similar manner to the step of inputting 105. This step of selecting 160 can be performed manually or automatically, as the result of a selection command transmitted to the computing system. The criteria for the selection can be arbitrary or follow an algorithmic resolution based upon information associated to each volatile molecule digital identifier, such as a cost value for each volatile molecule digital identifier.

The step of determination 165 is performed, for example, by a computing system configured to run a dedicated software. This step of determination 165 is designed to select a volatile molecule digital identifier fulfilling the same role as the at least two volatile molecule digital identifiers selected during the step of selection 160. The role, in this context, being defined by the impact of said volatile molecules upon at least two ORs. In other words, the OR impact signature of the identified volatile molecule should correspond to the OR impact signature of the selected volatile molecules.

This step of determination 165 may follow optimization rules such as discussed above for other steps of determination, 135 or 155.

The step of supplying 140 the result of the step of determination 165 corresponds to the step of supplying 140 discussed above.

In particular embodiments, such as the one shown in FIG. 1, the method 100 object of the present invention comprises, downstream of a step of determination, 135, 155 or 165, a step of estimation 170 of a quantity for at least one said volatile molecule digital identifier, said quantity being used in a downstream step of supplying 140.

The step of estimation 170 is performed, for example, by a computing system configured to run a dedicated software. This step of estimation 170 uses, for example, an impact function associating a quantity of volatile molecule to an activity level of an OR. Said impact function may be generic for all volatile molecules or specific to each volatile molecule.

Volatile molecules may compete for the binding of odorant receptors and may subsequently activate them. In these cases, the relative quantities of molecules, their respective binding coefficients and propensities to induce activation determine the total activation of an OR.

Single molecules that activate ORs do so with a sigmoidal dependence on the logarithm of the concentration of the molecule in contact with the receptor. To determine said dependence, a Person Skilled in the Art would contact a given molecule at different concentration levels and record the activation level of the OR. Ranging the activation levels ORs by increasing concentration of the molecule, and by using a sigmoid fit function, a resulting formula could be obtained to approximate, from any concentration value, a resulting activation level for the OR.

Multiple molecules that activate ORs do so as a nonlinear function of the ratios of their binding coefficients, the logarithm of the concentrations of the molecular species and their individual propensities to activate the receptor. In another mode of operation, two or more volatiles may bind the receptor non-competitively and unidirectionally or reciprocally influence the binding affinities and/or activation propensities of the other bound molecule. The total activity of the receptor is then a nonlinear combination of the logarithm of the concentrations, the binding coefficients, the propensities for activation and the coefficients of modulation of binding coefficients and activation propensities. In all of these cases the resulting total activity can be measured directly in experimental assays and used to determine the function associating the quantity of volatile molecules to the total activity.

Such a function is used by means of regression to determine, from a determined impact, a quantity of the corresponding volatile molecule.

Such embodiments may trigger additional optimization rules based upon the quantity of volatile molecules, such as discussed above.

In particular embodiments, such as the one shown in FIG. 1, the method 100 object of the present invention comprises a step of inputting 175 quantity of at least one input volatile molecule, upon a computer interface, said quantity being used in the step of calculating 110, by a computing system, for at least one volatile molecule digital identifier of the formula, a value representative of an impact on an activity level of an odorant receptor.

The step of inputting 175 is performed, for example, in a similar manner to the step of inputting 105. In such a step of inputting 175 a value representative of a quantity of an input molecule, during the step of inputting 105, a numerical value is input, said value being representative of the quantity of said volatile molecule.

The step of calculating 110 may use an impact function associating a quantity of volatile molecule to an activity level of an OR. Said impact function may be generic for all volatile molecules or specific to each volatile molecule. The input quantity allows for the calculation of the impact of said volatile molecule, which in turn allows for the determination of the degree of perceivability of a tonality of the formula.

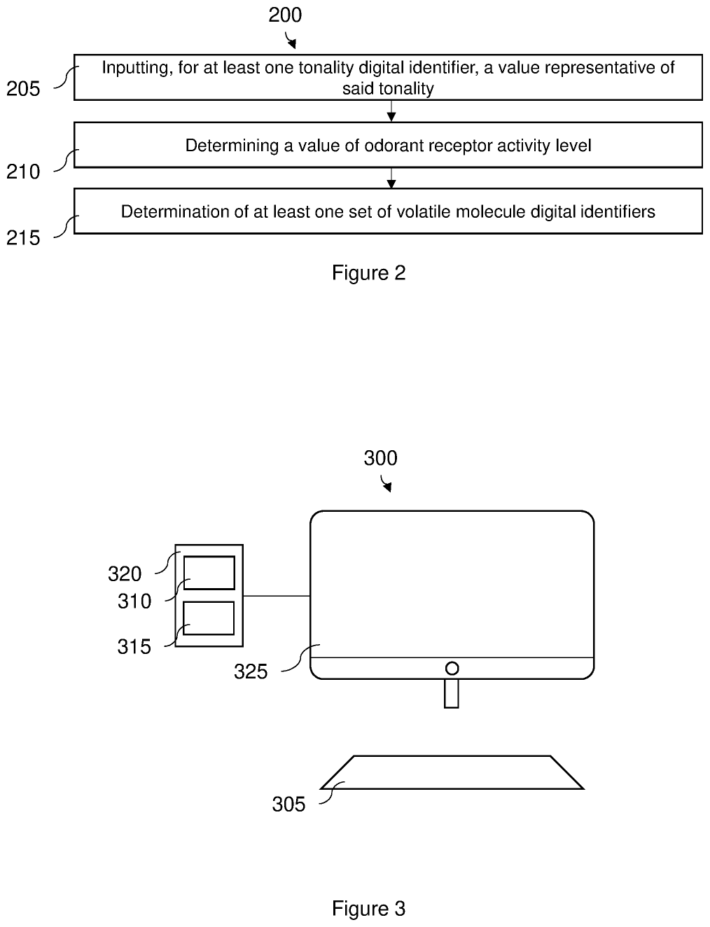

FIG. 2 shows a particular succession of steps of the method 100 object of the present invention. This formula determination method 200 comprises:
a step of inputting 205, for at least one tonality digital identifier, upon a computer interface, a value representative of the tonality of a composition resulting from a formula to be determined,
a step of determining 210, by a computing system, for the formula to be determined and as a function of at least one value representative of at least one tonality of said composition, a value of odorant receptor activity level for at least one odorant receptor digital identifier representative of said odorant receptor, each odorant receptor digital identifier being associated with one tonality digital identifier, said association being a one-to-one association, and
a step of determination 215 of a formula represented by at least a set of at least one volatile molecule digital identifier presenting a value representative of an impact on the activity level of said odorant receptor digital identifier equal to the determined value representative of the activity level on said odorant receptor, each volatile molecule digital identifier being associated with at least one odorant receptor digital identifier, said association being a many-to-many association.

The step of inputting 205 is performed in a similar manner to the step of inputting 105 described with regards to FIG. 1. This step can be manual or automatic, depending on the use case of the present invention. During this step of inputting 205, rather than inputting volatile molecule identifiers as in FIG. 1, a tonality digital identifier is input. Such a tonality digital identifier may correspond to the presence of a tonality within a formula or to a degree of perceivability of said tonality.

The step of determining 210 is performed, for example, by a computing system configured to run a dedicated software. This step of determining 210 is configured to perform in reverse of the step of determining 115 of FIG. 1, in that from a given tonality identifier input, an impact on OR activity may be determined. This step of determining 210 functions similarly to the step of calculation 130 of a modification of total activity level, only not limited to the modification but rather the entire impact evaluation.

The step of determination 215 is performed, for example, by a computing system configured to run a dedicated software. This step of determination 215 functions in a similar manner to the step of determination 135.

The output of the step of determination 215 may be a computer interface. Such an output is similar to the step of supplying 140 disclosed in regard to FIG. 1.

In particular embodiments, the step of supplying 140 is configured to supply the corresponding data to a corresponding actuator, such as a formula assembly means configured to assemble the formula corresponding to the volatile molecule supplied in the quantities determined (or in standard quantities). In such embodiments, the assembly means may comprise samples of the volatile molecules and reactors configured to receive all or part of said samples, the volatile molecules being fed to the reactor as a function of the formula resulting from the method, 100 or 200.

FIG. 3 shows a particular embodiment of the system 300 object of the present invention. This composition tonality determination system 300 comprises:
a means of inputting 305 at least one volatile molecule digital identifier, said volatile molecule digital identifier being representative of a fragrant volatile molecule, said input defining a formula,
a means of calculating 310, for at least one volatile molecule digital identifier of the formula, a value representative of an impact on an activity level of each said molecule on an activity level of an odorant receptor, represented by an odorant receptor digital identifier, each volatile molecule digital identifier being associated with at least one odorant receptor digital identifier, said association being a many-to-many association and
a means of determining 315, for the composition and as a function of at least one odorant receptor activity level impact calculated and a value representative of an odorant receptor activation threshold, a value representative of at least one tonality forming a composition, each odorant receptor digital identifier being associated with one tonality digital identifier, said association being a one-to-one association.

The particular embodiments of the means of the system 300 have been described in relation to the functional steps undertaken in the description of FIGS. 1 and 2 discussed above.

The means of inputting 305 is, for example, a keyboard, mouse and/or touchscreen adapted to interact with a computing system in such a way to collect user input. In variants, the means of inputting 305 are logical in nature, such as a network port of a computing system configured to receive an input command transmitted electronically. Such an input means 305 may be associated to a GUI shown to a user or an API (Application programming interface).

The means of calculating 310 and determining 315 can be, for example, a computing system configured to run a dedicated software in charge of executing the corresponding algorithms.

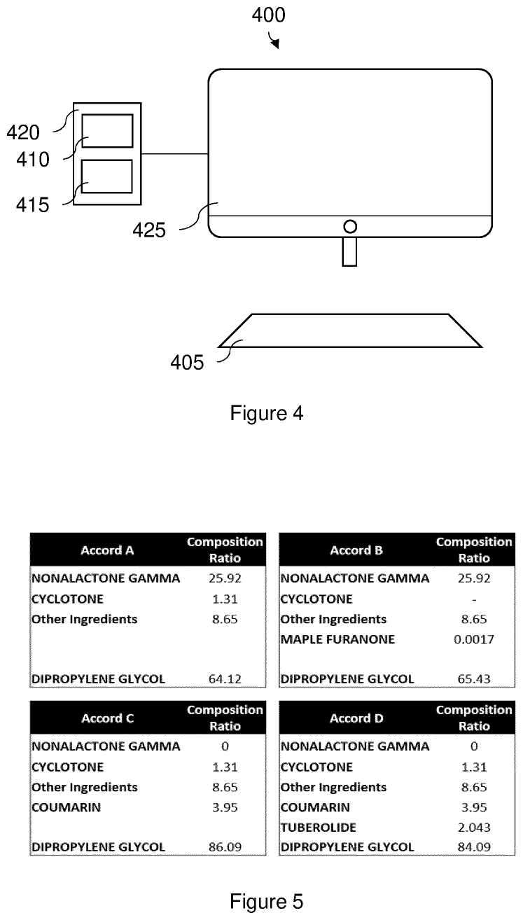

FIG. 4 shows a particular embodiment of the system 400 object of the present invention. This fragrance formula determination system 400 comprises:
a means of inputting 405, for at least one tonality digital identifier, upon a computer interface, a value representative of the tonality of a composition resulting from a formula to be determined,
a means of determining 410, by a computing system, for the formula to be determined and as a function of at least one value representative of at least one tonality of said composition, a value of odorant receptor activity level for at least one odorant receptor digital identifier representative of said odorant receptor, each odorant receptor digital identifier being associated with one tonality digital identifier, said association being a one-to-one association, and
a means of determination 415 of a formula represented by at least a set of at least one volatile molecule digital identifier presenting a value representative of an impact on the activity level of said odorant receptor digital identifier equal to the determined value representative of the activity level on said odorant receptor, each volatile molecule digital identifier being associated with at least one odorant receptor digital identifier, said association being a many-to-many association.

The means of inputting 405 are similar to the means of inputting 305 described in regards of FIG. 3.

The means of determining 410 and determination 415 can be, for example, a computing system configured to run a dedicated software in charge of executing the corresponding algorithms.

It should be understood from the previous description that a formula, defined by a selection of at least one volatile molecule, can be encoded into a set of OR indicators activated, said ORs corresponding to the tonalities perceived. Such an encoding system provides several uses in terms of formula optimization or performance prediction. FIGS. 5 to 11 provide an example of OR activity modeling and the impact on tonality prediction.

By way of example, in FIG. 5, a perfumery accord (accord A) is shown containing ingredients activating odorant receptors OR8B3 (contributing a hay tonality), OR8D1 (contributing a celery tonality) and OR5AU1 (contributing a lactonic coconut tonality) along with three accords in which small modifications were introduced (accords B-D). Ingredients that do not activate any of the three target ORs were aggregated for simplification and described as “other ingredients” in FIG. 5. Using receptor screening data for each OR (as described in Example 1 of U.S. 62/911,096), alternative activators can be used in replacement of an ingredient present in an original formula. This approach allows to prioritize the use of favored ingredients for the optimization of multiple factors including decreasing cost, increasing biodegradability, or using compounds with environmentally friendly properties.

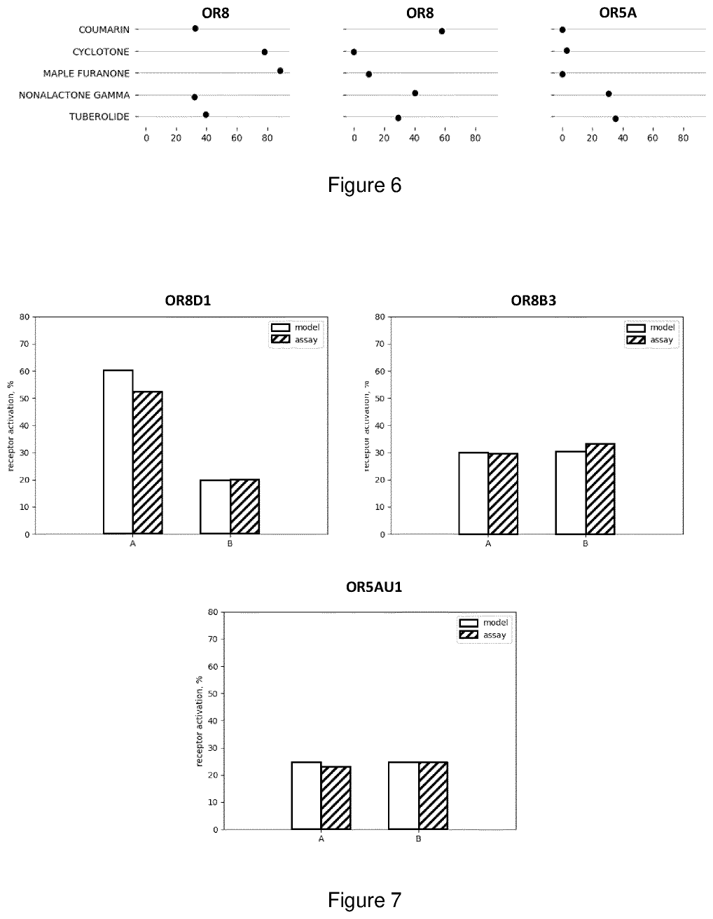

The computing system was used to determine the total receptor activity level of the accords shown in FIG. 5 from non-linear combinations of the empirically determined information on individual volatile molecules shown in FIG. 6 and verified to match empirical mixture responses as shown in FIGS. 7 & 9. Activity levels can be normalized to the maximal cellular response to Forskolin, a known pharmacological transduction cascade activator and serves as anchor point for data comparison purposes. The accord activity predicted by the model were compared to cell-based data generated with the same accords (cell-based assay as described in Example 2 of U.S. 62/911,096). Congruent activity levels were observed between model and in vitro data, validating the model. The resulting activity predictions between original and modified accords were used to make sensory predictions based on OR activity changes (i.e. more or less active) and inferring the corresponding tonality change (i.e. more or less intense). The sensory predictions were validated by perfumery expert sensory panel asked to perform a blind evaluation of the three tonalities of interest: hay, celery and lactonic coconut, for each accord.

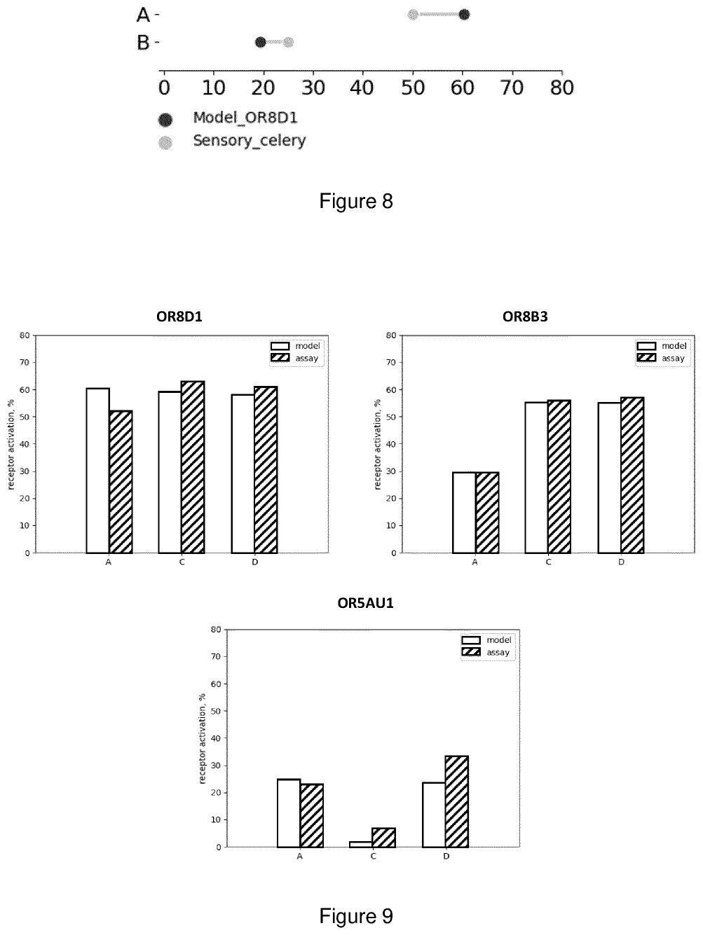

The case of a composition in which cyclotone was removed and replaced with maple furanone with respect to OR8D1 activity is shown in FIG. 5 (compare accord A and B). The target activity level was set to be lower for OR8D1 versus original. FIG. 7 shows the predicted activity level from the model in comparison to the in vitro validation for the receptor, normalized to 100% Forskolin response. A corresponding reduction in the perception of the celery tonality was predicted which was confirmed by the expert panelist evaluation as summarized in FIG. 8.

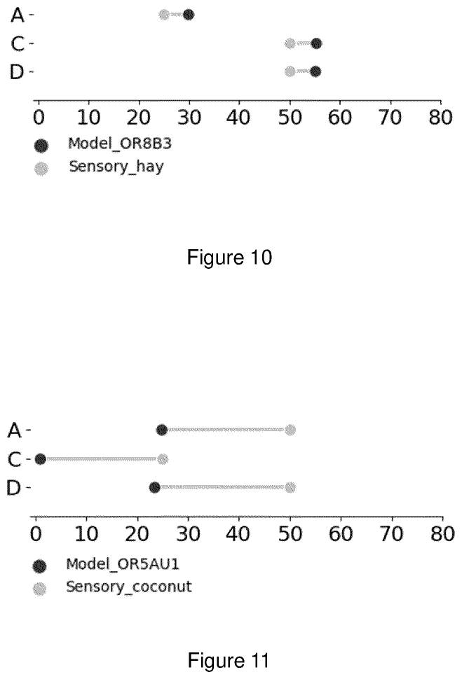

The case of a composition in which nonalactone was removed and replaced with coumarin with respect to OR8B3 and OR5AU1 activity is shown in FIG. 5 (compare accord A and C). In this case, the target activity level was set to be higher for OR8B3 and null for OR5AU1 versus original. FIG. 9 shows the predicted activity level from the model in comparison to the in vitro validation for both receptors, normalized to 100% Forskolin response. A corresponding increase in the perception of the hay and loss of the lactonic coconut tonalities were predicted which was confirmed by the expert panelist evaluation as summarized in FIG. 10.

The case of a composition in which nonalactone was replaced with coumarin, and tuberolide was added to produce desired OR8B3 and OR5AU1 activity is shown in FIG. 5 (compare accord A, C and D). The target activity level was set to be the same at OR8B3 for accord D versus accord C and restored to the level of accord A by accord D on OR5AU1. FIG. 9 shows the predicted activity level from the model in comparison to the in vitro validation for both receptors, normalized to 100% Forskolin response. A corresponding recovery of the perception of the coconut tonality due to the restored OR5AU1 activity was predicted along with a maintenance of the increased perception of the hay tonality which was confirmed by the expert panelist evaluation as summarized in FIG. 11.

These perfumery accord alteration examples capture the system's ability to model receptor activity for complex mixtures before and after removal and replacements of compounds using solely single compound input data (as obtained in Example 1-3 of U.S. 62/911,096). The ability of the system to predict sensory outcome based on calculated or measured receptor activity allows for maintaining the perception of a specific desired tonality using a different composition; adjusting a compound concentration or ratio in the mixture to decrease or increase a desired tonality; and for adding a compound to rebalance a composition by restoring a desired tonality lost during alterations. It is made clear by these examples that either the desired set of perceived tonalities could be set in the computing system (representing the target tonalities) and the possible sets of compounds and their concentrations determined that would yield the required total OR activities, or conversely that the composition could be altered as required and the resulting impact on the perceived tonalities could be determined by calculating the resulting OR activities, or further still, that a desired or measured set of receptor activity levels could be input and both the resulting perceived tonalities and plausible sets of compositions calculated.

1. A composition tonality determination method comprising:
a step of inputting at least one volatile molecule digital identifier, upon a computer interface, said volatile molecule digital identifier being representative of a fragrant volatile molecule, said input defining a formula,
a step of calculating, by a computing system, for at least one volatile molecule digital identifier of the formula, a value representative of an impact of each said molecule on an activity level of an odorant receptor, represented by an odorant receptor digital identifier, each volatile molecule digital identifier being associated with at least one odorant receptor digital identifier, said association being a many-to-many association, and
a step of determining, by the computing system, for the formula and as a function of at least one odorant receptor activity level impact calculated and a value representative of an odorant receptor activation threshold, a value representative of at least one tonality forming a composition, each odorant receptor digital identifier being associated with one tonality digital identifier, said association being a one-to-one association.

2. The method according to claim 1, which further comprises, downstream of the step of calculating, a step of computing, by the computing system, for at least one odorant receptor, a total activity level as a function of at least one impact on an activity level calculated.

3. The method according to claim 1, which further comprises:
a step of modification modifying, upon the computer interface, a value representative of a fragrance tonality of the composition,
a step of second calculating, by the computing system, a modification of total activity level of at least one odorant receptor,
a step of second determining, by the computing system, a set of at least one volatile molecule digital identifier presenting a total activity level impact value equal to the total activity level modification calculated, and
a step of supplying said set to a computer interface.

4. The method according to claim 3, in which each tonality is associated to a tonality digital identifier, said method further comprising a step of automatic tonality selection as a function of the result of comparing the tonality digital identifiers of the composition to a set of at least one predetermined tonality digital identifier, the step of modifying the value representative of the fragrance of said selected tonality identifiers of the composition being configured to reduce the value of said selected tonality identifiers to a reduced value.

5. The method according to claim 3, in which the value representative of the impact of the molecule, represented by the corresponding volatile molecule digital identifier, on an activity level of an odorant receptor is representative of one or more of:
the activation of said odorant receptor,
the deactivation of said odorant receptor,
the enhancement of said odorant receptor, or
the inhibition of said odorant receptor.

6. The method according to claim 5, in which the step of second determining is configured to provide at least one volatile molecule digital identifier presenting a value representative of an impact on the activity of an odorant receptor associated to a tonality modified during the step of modifying, said impact corresponding either to the enhancement of said odorant receptor or to the inhibition of said odorant receptor.

7. The method according to claim 3, which further comprises:
a step of selecting a volatile molecule digital identifier of the formula, upon the computer interface, and
a step of, third determining, by the computing system, at least a set of at least one volatile molecule digital identifier presenting a value representative of an impact on an activity level of a odorant receptor equal to the value representative of the impact on the activity level on said odorant receptor of the selected volatile molecule digital identifier, and
wherein the step of supplying further comprises supplying said set to the computer interface.

8. The method according to claim 7, which further comprises:
a step of second selecting at least two volatile molecule digital identifiers of the formula, at least two said volatile molecules being associated with the activation of at least two distinct odorant receptors, comprising target receptors
a step of fourth determining one volatile molecule digital identifier presenting a value representative of an impact on an activity level of each target receptor equal to the value representative of the impact on the activity level on said odorant receptor of the selected volatile molecule digital identifiers, and
wherein the step of supplying further comprises supplying the determined volatile molecule digital identifier to the computer interface.

9. The method according to claim 8, which comprises, downstream of the second determining, the third determining, or the fourth determining, a step of estimating a quantity for at least one said volatile molecule digital identifier, said quantity being used in the downstream step of supplying.

10. The method according to claim 7, in which the second determining or the third determining comprises executing an optimization rule which is at least one of the following:
a first rule in which a determined set of at least one volatile molecule digital identifier is configured to present a minimal number of volatile molecule digital identifiers,
a second rule in which a determined set of at least one volatile molecule digital identifier is configured to present a minimal total quantity indicator of corresponding volatile molecule digital identifiers,
a third rule in which a determined set of at least one volatile molecule digital identifier is configured to present a minimal number of negative impact volatile molecule digital identifiers,
a fourth rule in which at least one volatile molecule digital identifier is associated to a volatile molecule delivery capacity indicator, said rule being configured to adapt a determined set of at least one volatile molecule digital identifier as a function of said volatile molecule delivery capacity indicator for the volatile molecule digital identifiers in the set,
a fifth rule in which a set of at least two volatile molecule digital identifier is configured to minimize the overlap of odorant receptors activated by said set of molecules,
a sixth rule in which a set of at least two volatile molecule digital identifier is configured to minimize the modulation of the odorant receptors activated by said set of molecules,
a seventh rule in which determined set of at least one volatile molecule digital identifier is configured to maximize the negative modulation or inhibition of odorant receptor linked to a predetermined undesired olfactory tonality,
an eighth rule in which determined set of at least one volatile molecule digital identifier is configured to maximize the positive modulation or enhancement of odorant receptor linked to a predetermined desired olfactory tonality, or
a ninth rule in which determined set of at least one volatile molecule digital identifier is configured to minimize or maximize at least one cost function, each volatile molecule being associated to at least one value representative of cost.

11. The method according to claim 1, which further comprises a step of second inputting a quantity of at least one input volatile molecule, upon the computer interface, said quantity being used in the step of calculating, by the computing system, for at least one volatile molecule digital identifier of the formula, a value representative of an impact on an activity level of an odorant receptor.

12. The method according to claim 8, in which at least one volatile molecule digital identifier is associated to a volatile molecule delivery capacity indicator, at least one of steps of calculating second determining, third determining, or fourth determining being performed as a function of the volatile molecule delivery capacity indicator associated to at least one input volatile molecule digital identifier.

13. The method according to claim 12, in which the volatile molecule delivery capacity indicator is representative of a substrate upon which the associated volatile molecule is located.

14. A formula determination method comprising:
a step of inputting, for at least one tonality digital identifier, upon a computer interface, a value representative of the tonality of a composition resulting from a formula to be determined,
a step of determining, by a computing system, for the formula to be determined and as a function of at least one value representative of at least one tonality of said composition, a value of odorant receptor activity level for at least one odorant receptor digital identifier representative of said odorant receptor, each odorant receptor digital identifier being associated with at least one tonality digital identifier, said association being a one-to-one association, and
a step second determining a formula represented by at least a set of at least one volatile molecule digital identifier presenting a value representative of an impact on the activity level of said odorant receptor digital identifier equal to the determined value representative of the activity level on said odorant receptor, each volatile molecule digital identifier being associated with at least one odorant receptor digital identifier, said association being a many-to-many association.

15. A composition tonality determination system comprising
a means of inputting at least one volatile molecule digital identifier, said volatile molecule digital identifier being representative of a fragrant volatile molecule, said input defining a formula,
a means of calculating, for at least one volatile molecule digital identifier of the formula, a value representative of an impact on an activity level of each said molecule on an activity level of an odorant receptor, represented by an odorant receptor digital identifier, each volatile molecule digital identifier being associated with at least one odorant receptor digital identifier, said association being a many-to-many association, and
a means of determining, for the composition and as a function of at least one odorant receptor activity level impact calculated and a value representative of an odorant receptor activation threshold, a value representative of at least one tonality forming a composition, each odorant receptor digital identifier being associated with one tonality digital identifier, said association being a one-to-one association.

16. A formula determination system comprising:
a means of inputting, for at least one tonality digital identifier, upon a computer interface, a value representative of the tonality of a composition resulting from a formula to be determined,
a means of determining, by a computing system, for the formula to be determined and as a function of at least one value representative of at least one tonality of said composition, a value of odorant receptor activity level for at least one odorant receptor digital identifier representative of said odorant receptor, each odorant receptor digital identifier being associated with one tonality digital identifier, said association being a one-to-one association, and
a means of second determining a formula represented by at least a set of at least one volatile molecule digital identifier presenting a value representative of an impact on the activity level of said odorant receptor digital identifier equal to the determined value representative of the activity level on said odorant receptor, each volatile molecule digital identifier being associated with at least one odorant receptor digital identifier, said association being a many-to-many association.

17. The method of claim 4, wherein the reduced value is zero.


SEQUENCE LISTING (31 sequences)

SEQ ID NO: 1 - DNA, 24 bp, Artificial Sequence
  Tag
gattacaaggacgacgacgataag

SEQ ID NO: 2 - protein, 8 aa, artificial
  synthesized
DYKDDDDK

SEQ ID NO: 3 - DNA, 60 bp, Artificial Sequence
  synthesized
atgaacgggaccgagggcccaaacttctacgtgcctttctccaacaagacgggcgtggtg

SEQ ID NO: 4 - protein, 20 aa, Artificial Sequence
  Lucy tag
MNGTEGPNFYVPFSNKTGVV

SEQ ID NO: 5 - DNA, 51 bp, Artificial Sequence
  synthesized
atgagaccccagatcctgctgctcctggccctgctgaccctaggcctggct

SEQ ID NO: 6 - protein, 17 aa, Artificial Sequence
  synthesized
MRPQILLLLALLTLGLA

SEQ ID NO: 7 - protein, 10 aa, Artificial Sequence
  motif
MAYDRYVAIC

SEQ ID NO: 8 - protein, 7 aa, Artificial Sequence
  motif-2
FSTCSSH

SEQ ID NO: 9 - protein, 8 aa, Artificial Sequence
  motif-3
PMLNPFIY

SEQ ID NO: 10 - DNA, 948 bp, homo sapiens
atggaaattgtctccacaggaaacgaaactattactgaatttgtcctccttggcttctatgacatccctgaactgcatttcttgttttttattgtattcactgctgtctatgtcttcatcatcatagggaatatgctgattattgtagcagtggttagctcccagaggctccacaaacccatgtatattttcttggcgaatctgtccttcctggatattctctacacctccgcagtgatgccaaaaatgctggagggcttcctgcaagaagcaactatctctgtggctggttgcttgctccagttctttatcttcggctctctagccacagctgaatgcttactgctggctgtcatggcatatgaccgctacctggcaatttgctacccactccactacccactcctgatggggcccagacggtacatggggctggtggtcacaacctggctctctggatttgtggtagatggactggttgtggccctggtggcccagctgaggttctgtggccccaaccacattgaccagttttactgtgactttatgcttttcgtgggcctggcttgctcggatcccagagtggctcaggtgacaactctcattctgtctgtgttctgcctcactattccttttggactgattctgacatcttatgccagaattgtggtggcagtgctgagagttcctgctggggcaagcaggagaagggctttctccacatgctcctcccacctagctgtagtgaccacattctatggaacgctcatgatcttttatgttgcaccctctgctgtccattcccagctcctctccaaggtcttctccctgctctacactgtggtcacccctctcttcaatcctgtgatctataccatgaggaacaaggaggtgcatcaggcacttcggaagattctctgtatcaaacaaactgaaacacttgattga

SEQ ID NO: 11 - protein, 315 aa, homo sapiens
MEIVSTGNETITEFVLLGFYDIPELHFLFFIVFTAVYVFIIIGNMLIIVAVVSSQRLHKPMYIFLANLSFLDILYTSAVMPKMLEGFLQEATISVAGCLLQFFIFGSLATAECLLLAVMAYDRYLAICYPLHYPLLMGPRRYMGLVVTTWLSGFVVDGLVVALVAQLRFCGPNHIDQFYCDFMLFVGLACSDPRVAQVTTLILSVFCLTIPFGLILTSYARIVVAVLRVPAGASRRRAFSTCSSHLAVVTTFYGTLMIFYVAPSAVHSQLLSKVFSLLYTVVTPLFNPVIYTMRNKEVHQALRKILCIKQTETLD

SEQ ID NO: 12 - DNA, 942 bp, homo sapiens
atgctggctagaaacaactccttagtgactgaatttattcttgctggattaacagatcatccagagttccagcaacccctctttttcctgtttctagtggtctacattgtcaccatggtaggcaaccttggcttgatcattcttttcggtctaaattctcacctccacacaccaatgtactatttcctcttcaatctctccttcattgatctctgttactcctctgttttcactcccaaaatgctaatgaactttgtatcaaaaaagaatattatctcctatgttgggtgcatgactcagctgtttttctttctcttttttgtcatctctgaatgttacatgttgacctcaatggcatatgatcgctatgtggccatctgtaatccattgctgtataaggtcaccatgtcccatcaggtctgttctatgctcacttttgctgcttacataatgggattggctggagccacggcccacaccgggtgcatgcttagactcaccttctgcagtgctaatatcatcaaccattacttgtgtgacatactccccctcctccagctttcctgcaccagcacctatgtcaacgaggtggttgttctcattgttgtgggtattaatatcatggtacccagttgtaccatcctcatttcttatgttttcattgtcactagcattcttcatatcaaatccactcaaggaagatcaaaagccttcagtacttgtagctctcatgtcattgctctgtctctgttttttgggtcagcggcattcatgtatattaaatattcttctggatctatggagcagggaaaagtttcttctgttttctacactaatgtggtgcccatgctcaatcctctcatctacagtttgaggaacaaggatgtcaaagttgcactgaggaaagctctgattaaaattcagagaagaaatatattctaa

SEQ ID NO: 13 - protein, 313 aa, homo sapiens
MLARNNSLVTEFILAGLTDHPEFQQPLFFLFLVVYIVTMVGNLGLIILFGLNSHLHTPMYYFLFNLSFIDLCYSSVFTPKMLMNFVSKKNIISYVGCMTQLFFFLFFVISECYMLTSMAYDRYVAICNPLLYKVTMSHQVCSMLTFAAYIMGLAGATAHTGCMLRLTFCSANIINHYLCDILPLLQLSCTSTYVNEVVVLIVVGINIMVPSCTILISYVFIVTSILHIKSTQGRSKAFSTCSSHVIALSLFFGSAAFMYIKYSSGSMEQGKVSSVFYTNVVPMLNPLIYSLRNKDVKVALRKALIKIQRRNIF

SEQ ID NO: 14 - DNA, 936 bp, homo sapiens
atgaaaggggcaaacctgagccaagggatggagtttgagctcttgggcctcaccactgacccccagctccagaggctgctcttcgtggtgttcctgggcatgtacacagccactctgctggggaacctggtcatgttcctcctgatccatgtgagtgccaccctgcacacacccatgtactccctcctgaagagcctctccttcttggatttctgctactcctccacggttgtgccccagaccctggtgaacttcttggccaagaggaaagtgatctcttattttggctgcatgactcagatgttcttctatgcgggttttgccaccagtgagtgctatctcatcgctgccatggcctatgaccgctatgccgctatttgtaaccccctgctctactcaaccatcatgtctcctgaggtctgtgcctcgctgattgtgggctcctacagtgcaggattcctcaattctcttatccacactggctgtatctttagtctgaaattctgcggtgctcatgtcgtcactcacttcttctgtgatgggccacccatcctgtccttgtcttgtgtagacacctcactgtgtgagatcctgctcttcatttttgctggtttcaaccttttgagctgcaccctcaccatcttgatctcctacttcttaattctcaacaccatcctgaaaatgagctcggcccagggcaggtttaaggcattttccacctgtgcatcccacctcactgccatctgcctcttctttggcacaacactttttatgtacctgcgccccaggtccagctactccttgacccaggaccgcacagttgctgtcatctacacagtggtgatcccagtgctgaaccccctcatgtactctttgagaaacaaggatgtgaagaaagctttaataaaggtttggggtaggaaaacaatggaatga

SEQ ID NO: 15 - protein, 311 aa, homo sapiens
MKGANLSQGMEFELLGLTTDPQLQRLLFVVFLGMYTATLLGNLVMFLLIHVSATLHTPMYSLLKSLSFLDFCYSSTVVPQTLVNFLAKRKVISYFGCMTQMFFYAGFATSECYLIAAMAYDRYAAICNPLLYSTIMSPEVCASLIVGSYSAGFLNSLIHTGCIFSLKFCGAHVVTHFFCDGPPILSLSCVDTSLCEILLFIFAGFNLLSCTLTILISYFLILNTILKMSSAQGRFKAFSTCASHLTAICLFFGTTLFMYLRPRSSYSLTQDRTVAVIYTVVIPVLNPLMYSLRNKDVKKALIKVWGRKTME

SEQ ID NO: 16 - DNA, 927 bp, homo sapiens
atgaccatggaaaattattctatggcagctcagtttgtcttagatggtttaacacagcaagcagagctccagctgcccctcttcctcctgttcctgggaatctatgtggtcacagtagtgggcaacctgggcatgattctcctgattgcagtcagccctctacttcacacccccatgtactatttcctcagcagcttgtccttcgtcgatttctgctattcctctgtcattactcccaaaatgctggtgaacttcctaggaaagaagaatacaatcctttactctgagtgcatggtccagctctttttctttgtggtctttgtggtggctgagggttacctcctgactgccatggcatatgatcgctatgttgccatctgtagcccactgctttataatgcgatcatgtcctcatgggtctgctcactgctagtgctggctgccttcttcttgggctttctctctgccttgactcatacaagtgccatgatgaaactgtccttttgcaaatcccacattatcaaccattacttctgtgatgttcttcccctcctcaatctctcctgctccaacacacacctcaatgagcttctactttttatcattgcggggtttaacaccttggtgcccaccctagctgttgctgtctcctatgccttcatcctctacagcatccttcacatccgctcctcagagggccggtccaaagcttttggaacatgcagctctcatctcatggctgtggtgatcttctttgggtccattaccttcatgtatttcaagcccccttcaagtaactccctggaccaggagaaggtgtcctctgtgttctacaccacggtgatccccatgctgaaccctttaatatacagtctgaggaataaggatgtgaagaaagcattaaggaaggtcttagtaggaaaatga

SEQ ID NO: 17 - protein, 308 aa, homo sapiens
MTMENYSMAAQFVLDGLTQQAELQLPLFLLFLGIYVVTVVGNLGMILLIAVSPLLHTPMYYFLSSLSFVDFCYSSVITPKMLVNFLGKKNTILYSECMVQLFFFVVFVVAEGYLLTAMAYDRYVAICSPLLYNAIMSSWVCSLLVLAAFFLGFLSALTHTSAMMKLSFCKSHIINHYFCDVLPLLNLSCSNTHLNELLLFIIAGFNTLVPTLAVAVSYAFILYSILHIRSSEGRSKAFGTCSSHLMAVVIFFGSITFMYFKPPSSNSLDQEKVSSVFYTTVIPMLNPLIYSLRNKDVKKALRKVLVGK

SEQ ID NO: 18 - DNA, 936 bp, homo sapiens
atgactgggggaggaaatattacagaaatcacctatttcatcctgctgggattctcagattttcccaggatcataaaagtgctcttcactatattcctggtgatctacattacatctctggcctggaacctctccctcattgttttaataaggatggattcccacctccatacacccatgtatttcttcctcagtaacctgtccttcatagatgtctgctatatcagctccacagtccccaagatgctctccaacctcttacaggaacagcaaactatcacttttgttggttgtattattcagtactttatcttttcaacgatgggactgagtgagtcttgtctcatgacagccatggcttatgatcgttatgctgccatttgtaaccccctgctctattcatccatcatgtcacccaccctctgtgtttggatggtactgggagcctacatgactggcctcactgcttctttattccaaattggtgctttgcttcaactccacttctgtgggtctaatgtcatcagacatttcttctgtgacatgccccaactgttaatcttgtcctgtactgacactttctttgtacaggtcatgactgctatattaaccatgttctttgggatagcaagtgccctagttatcatgatatcctatggctatattggcatctccatcatgaagatcacttcagctaaaggcaggtccaaggcattcaacacctgtgcttctcatctaacagctgtttccctcttctatacatcaggaatctttgtctatttgagttccagctctggaggttcttcaagctttgacagatttgcatctgttttctacactgtggtcattcccatgttaaatcccttgatttacagtttgaggaacaaagaaattaaagatgccttaaagaggttgcaaaagagaaagtgctgctga

SEQ ID NO: 19 - protein, 311 aa, homo sapiens
MTGGGNITEITYFILLGFSDFPRIIKVLFTIFLVIYITSLAWNLSLIVLIRMDSHLHTPMYFFLSNLSFIDVCYISSTVPKMLSNLLQEQQTITFVGCIIQYFIFSTMGLSESCLMTAMAYDRYAAICNPLLYSSIMSPTLCVWMVLGAYMTGLTASLFQIGALLQLHFCGSNVIRHFFCDMPQLLILSCTDTFFVQVMTAILTMFFGIASALVIMISYGYIGISIMKITSAKGRSKAFNTCASHLTAVSLFYTSGIFVYLSSSSGGSSSFDRFASVFYTVVIPMLNPLIYSLRNKEIKDALKRLQKRKCC

SEQ ID NO: 20 - DNA, 951 bp, homo sapiens
atgggaaaaccaggcagagtgaaccaaaccactgtttcagacttcctccttttaggactctctgagcggccagaggagcagcctcttctgtttggcatcttccttggcatgtacctggtcaccatggtggggaacctgctcattatcctggccatcagctctgacccacacctccatactcccatgtacttctttctggccaacctgtcattaactgatgcctgtttcacttctgcctccatccccaaaatgctggccaacattcatacccagagtcagatcatctcctattctgggtgtcttgcacagctatatttcctccttatgtttggtggccttgacaactgcctgctggctgtgatggcatatgaccgctatgtggccatctgccaaccactccattacagcacatctatgagtccccagctctgtgcactaatgctgggtgtgtgctgggtgctaaccaactgtcctgccctgatgcacacactgttgctgacccgcgtggctttctgtgcccagaaagccatccctcatttctactgtgatcccagtgctctcctgaagcttgcctgctcagatacccatgtaaacgagctgatgatcatcaccatgggcttgctgttcctcactgttcccctcctgctgatcgtcttctcctatgtccgcattttctgggctgtgtttggcatctcatctcctggagggagatggaaggccttctctacctgtggttctcatctcacggtggttctgctcttctatgggtctcttatgggtgtgtatttacttcctccatcaacttactctacagagagggaaagtagggctgctgttctctatatggtgattattcccatgctaaacccattcatttatagcttgaggaacagagacatgaaggaggctttgggtaaactttttgtcagtggaaaaacattctttttatga

SEQ ID NO: 21 - protein, 316 aa, homo sapiens
MGKPGRVNQTTVSDFLLLGLSERPEEQPLLFGIFLGMYLVTMVGNLLIILAISSDPHLHTPMYFFLANLSLTDACFTSASIPKMLANIHTQSQIISYSGCLAQLYFLLMFGGLDNCLLAVMAYDRYVAICQPLHYSTSMSPQLCALMLGVCWVLTNCPALMHTLLLTRVAFCAQKAIPHFYCDPSALLKLACSDTHVNELMIITMGLLFLTVPLLLIVFSYVRIFWAVFGISSPGGRWKAFSTCGSHLTVVLLFYGSLMGVYLLPPSTYSTERESRAAVLYMVIIPMLNPFIYSLRNRDMKEALGKLFVSGKTFFL

SEQ ID NO: 22 - DNA, 948 bp, homo sapiens
atgtccataaccaaagcctggaacagctcatcagtgaccatgttcatcctcctgggattcacagaccatccagaactccaggccctcctctttgtgaccttcctgggcatctatcttaccaccctggcctggaacctggccctcatttttctgatcagaggtgacacccatctgcacacacccatgtacttcttcctaagcaacttatctttcattgacatctgctactcttctgctgtggctcccaatatgctcactgacttcttctgggagcagaagaccatatcatttgtgggctgtgctgctcagttttttttctttgtcggcatgggtctgtctgagtgcctcctcctgactgctatggcatacgaccgatatgcagccatctccagcccccttctctaccccactatcatgacccagggcctctgtacacgcatggtggttggggcatatgttggtggcttcctgagctccctgatccaggccagctccatatttaggcttcacttttgcggacccaacatcatcaaccacttcttctgcgacctcccaccagtcctggctctgtcttgctctgacaccttcctcagtcaagtggtgaatttcctcgtggtggtcactgtcggaggaacatcgttcctccaactccttatctcctatggttacatagtgtctgcggtcctgaagatcccttcagcagagggccgatggaaagcctgcaacacgtgtgcctcgcatctgatggtggtgactctgctgtttgggacagcccttttcgtgtacttgcgacccagctccagctacttgctaggcagggacaaggtggtgtctgttttctattcattggtgatccccatgctgaaccctctcatttacagtttgaggaacaaagagatcaaggatgccctgtggaaggtgttggaaaggaagaaagtgttttcttag

SEQ ID NO: 23 - protein, 315 aa, homo sapiens
MSITKAWNSSSVTMFILLGFTDHPELQALLFVTFLGIYLTTLAWNLALIFLIRGDTHLHTPMYFFLSNLSFIDICYSSAVAPNMLTDFFWEQKTISFVGCAAQFFFFVGMGLSECLLLTAMAYDRYAAISSPLLYPTIMTQGLCTRMVVGAYVGGFLSSLIQASSIFRLHFCGPNIINHFFCDLPPVLALSCSDTFLSQVVNFLVVVTVGGTSFLQLLISYGYIVSAVLKIPSAEGRWKACNTCASHLMVVTLLFGTALFVYLRPSSSYLLGRDKVVSVFYSLVIPMLNPLIYSLRNKEIKDALWKVLERKKVFS

SEQ ID NO: 24 - DNA, 930 bp, homo sapiens
atggaaccagagaatgacacagggatttcagaatttgttcttctgggactttctgaggaaccagaattgcagcccttcctctttgggctgtttctgtccatgtacctggtcactgtgctcgggaatctgctcatcatcctggccacaatctcagactcccacctccacacccccatgtacttcttcctctccaacctgtcctttgcagacatctgtttcatctccactacaatcccaaagatgctcattaacatccagacacagagcagagtcatcacctatgcaggctgcatcacccagatgtgcttttttgtactttttggagggttagacagcttactcctggctgtgatggcctatgatcggtttgtggccatctgtcatcctctgcactacacagtcatcatgaaccctcggctctgtggactcctggttctggcatcctggatgattgctgccctgaattccttgtcacaaagcttaatggtattgtggctgtccttctgcacagacttggaaatcccccactttttctgtgaacttaatcaggtcatccaccttgcctgttctgacacctttcttaatgacatggggatgtattttgcagcagggctgctggctggtggtccccttgtggggatcctttgctcttactctaagatagtttcctccatacgtgcaatctcatcagctcaggggaagtacaaggcattttccacctgtgcatcacacctctcagttgtctctttattttgttgtacgggcctaggtgtgtaccttacttctgctgcaacccacaactcacacacaagtgcaacagcctcagtgatgtacactgtggccacccccatgctgaacccctttatctacagtctgaggaataaagacataaagagggctctgaaaatgtccttcagaggaaagcaataa

SEQ ID NO: 25 - protein, 309 aa, homo sapiens
MEPENDTGISEFVLLGLSEEPELQPFLFGLFLSMYLVTVLGNLLIILATISDSHLHTPMYFFLSNLSFADICFISTTIPKMLINIQTQSRVITYAGCITQMCFFVLFGGLDSLLLAVMAYDRFVAICHPLHYTVIMNPRLCGLLVLASWMIAALNSLSQSLMVLWLSFCTDLEIPHFFCELNQVIHLACSDTFLNDMGMYFAAGLLAGGPLVGILCSYSKIVSSIRAISSAQGKYKAFSTCASHLSVVSLFCCTGLGVYLTSAATHNSHTSATASVMYTVATPMLNPFIYSLRNKDIKRALKMSFRGKQ

SEQ ID NO: 26 - DNA, 930 bp, homo sapiens
atgaagagaaagaacttcacagaagtgtcagaattcattttcttgggattttctagctttggaaagcatcagataaccctctttgtggttttcctaactgtctacattttaactctggttgctaacatcatcattgtgactatcatctgcattgaccatcatctccacactcccatgtatttcttcctaagcatgctggctagttcagagacggtgtacacactggtcattgtgccacgaatgcttttgagcctcatttttcataaccaacctatctccttggcaggctgtgctacacaaatgttcttttttgttatcttggccactaataattgcttcctgcttactgcaatggggtatgaccgctatgtggccatctgcagacccctgagatacactgtcatcatgagcaagggactatgtgcccagctggtgtgtgggtcctttggcattggtctgactatggcagttctccatgtgacagccatgttcaatttgccgttctgtggcacagtggtagaccacttcttttgtgacatttacccagtcatgaaactttcttgcattgataccactatcaatgagataataaattatggtgtaagttcatttgtgatttttgtgcccataggcctgatatttatctcctatgtccttgtcatctcttccatccttcaaattgcctcagctgagggccggaagaagacctttgccacctgtgtctcccacctcactgtggttattgtccactgtggctgtgcctccattgcctacctcaagccgaagtcagaaagttcaatagaaaaagaccttgttctctcagtgacgtacaccatcatcactcccttgctgaaccctgttgtttacagtctgagaaacaaggaggtaaaggatgccctatgcagagttgtgggcagaaatatttcttaa

SEQ ID NO: 27 - protein, 309 aa, homo sapiens
MKRKNFTEVSEFIFLGFSSFGKHQITLFVVFLTVYILTLVANIIIVTIICIDHHLHTPMYFFLSMLASSETVYTLVIVPRMLLSLIFHNQPISLAGCATQMFFFVILATNNCFLLTAMGYDRYVAICRPLRYTVIMSKGLCAQLVCGSFGIGLTMAVLHVTAMFNLPFCGTVVDHFFCDIYPVMKLSCIDTTINEIINYGVSSFVIFVPIGLIFISYVLVISSILQIASAEGRKKTFATCVSHLTVVIVHCGCASIAYLKPKSESSIEKDLVLSVTYTIITPLLNPVVYSLRNKEVKDALCRVVGRNIS

SEQ ID NO: 28 - DNA, 945 bp, homo sapiens
atggaaaaaagaaatctaacagttgtcagggaattcgtccttctgggacttcctagctcagcagagcagcagcacctcctgtctgtgctctttctctgtatgtatttagccaccaccttggggaacatgctcatcattgcgacgattggctttgactctcacctccattcccctatgtacttcttccttagtaacttggcctttgttgacatctgctttacgtcgactacagtcccccaaatggtagtgaatatcttgactggcaccaagactatctcttttgcaggctgcctcacccagctcttcttcttcgtttcttttgtgaatatggacagcctccttctgtgtgtgatggcgtatgatagatatgtggcgatttgccaccccttacattacaccgccagaatgaacctgtgcctttgtgtccagctagtggctggactgtggcttgttacttacctccacgccctcctgcatactgtcctaatagcacagctgtccttctgtgcctccaatatcatccatcatttcttctgtgatctcaatcctctcctgcagctctcttgctctgacgtctccttcaatgtaatgatcatttttgcagtaggaggtctattggctctcacgccccttgtctgtatcctcgtatcttatggacttatcttctccactgttctgaagatcacctctactcagggcaagcagagagctgtttccacctgcagctgccacctgtcagtggtggtgttgttttacggcacagccatcgccgtctatttcagcccttcatccccccatatgcctgagagcgacactctgtcaaccatcatgtattcaatggtggctccgatgctgaatcctttcatctataccctaaggaacagggatatgaagaggggacttcagaaaatgcttctcaagtgcacagtctttcagcagcaataa

SEQ ID NO: 29 - protein, 314 aa, homo sapiens
MEKRNLTVVREFVLLGLPSSAEQQHLLSVLFLCMYLATTLGNMLIIATIGFDSHLHSPMYFFLSNLAFVDICFTSTTVPQMVVNILTGTKTISFAGCLTQLFFFVSFVNMDSLLLCVMAYDRYVAICHPLHYTARMNLCLCVQLVAGLWLVTYLHALLHTVLIAQLSFCASNIIHHFFCDLNPLLQLSCSDVSFNVMIIFAVGGLLALTPLVCILVSYGLIFSTVLKITSTQGKQRAVSTCSCHLSVVVLFYGTAIAVYFSPSSPHMPESDTLSTIMYSMVAPMLNPFIYTLRNRDMKRGLQKMLLKCTVFQQQ

SEQ ID NO: 30 - DNA, 945 bp, homo sapiens
atggagaacaacacagaggtgactgaattcatccttgtggggttaactgatgacccagaactgcagatcccactcttcatagtcttccttttcatctacctcatcactctggttgggaacctggggatgattgaattgattctactggactcctgtctccacacccccatgtacttcttcctcagtaacctctccctggtggactttggttattcctcagctgtcactcccaaggtgatggtggggtttctcacaggagacaaattcatattatataatgcttgtgccacacaattcttcttctttgtagcctttatcactgcagaaagtttcctcctggcatcaatggcctatgaccgctatgcagcattgtgtaaacccctgcattacaccaccaccatgacaacaaatgtatgtgcttgcctggccataggctcctacatctgtggtttcctgaatgcatccattcatactgggaacactttcaggctctccttctgtagatccaatgtagttgaacactttttctgtgatgctcctcctctcttgactctctcatgttcagacaactacatcagtgagatggttattttttttgtggtgggattcaatgacctcttttctatcctggtaatcttgatctcctacttatttatatttatcaccatcatgaagatgcgctcacctgaaggacgccagaaggccttttctacttgtgcttcccaccttactgcagtttccatcttttatgggacaggaatctttatgtacttacgacctaactccagccatttcatgggcacagacaaaatggcatctgtgttctatgccatagtcattcccatgttgaatccactggtctacagcctgaggaacaaagaggttaagagtgcctttaaaaagactgtagggaaggcaaaggcctctataggattcatattttaa

SEQ ID NO: 31 - protein, 314 aa, homo sapiens
MENNTEVTEFILVGLTDDPELQIPLFIVFLFIYLITLVGNLGMIELILLDSCLHTPMYFFLSNLSLVDFGYSSAVTPKVMVGFLTGDKFILYNACATQFFFFVAFITAESFLLASMAYDRYAALCKPLHYTTTMTTNVCACLAIGSYICGFLNASIHTGNTFRLSFCRSNVVEHFFCDAPPLLTLSCSDNYISEMVIFFVVGFNDLFSILVILISYLFIFITIMKMRSPEGRQKAFSTCASHLTAVSIFYGTGIFMYLRPNSSHFMGTDKMASVFYAIVIPMLNPLVYSLRNKEVKSAFKKTVGKAKASIGFIF